(19) United States
(12) Patent Application Publication
(10) Pub. No.: US 2002/0155108 A1
(43) Pub. Date: Oct. 24, 2002

(54) Method for ex vivo loading of antigen presenting cells with antigen, and a vaccine comprising the loaded cells

(75) Inventor: Emilio Barbera-Guillem, Powell, OH (US)

Correspondence Address: BENESCH, FRIEDLANDER, COPLAN & ARONOFF LLP, ATTN: IP DEPARTMENT DOCKET CLERK, 2300 BP TOWER, 200 PUBLIC SQUARE, CLEVELAND, OH, 44114, US

(73) Assignee: BioCrystal, Ltd., Westerville, OH

(21) Appl. No.: 10/118,379

(22) Filed: Apr. 8, 2002

Related U.S. Application Data
(63) Continuation of application No. 09/303,701, filed on May 3, 1999.
(60) Provisional application No. 60/084,041, filed on May 4, 1998.

(51) Int. Cl. A61K 39/395; C12N 5/08

(52) U.S. Cl. 424/140.1; 424/145.1; 435/372

(57) ABSTRACT

Provided is a method for loading dendritic cells ex vivo which utilizes immune complex-mediated uptake of a soluble antigen by dendritic cells. The method involves coculturing target cells in the presence of, but not in physical contact with, expanded dendritic cells. Added to the coculture is human antibody against the soluble antigen(s), resulting in formation of immune complexes. The immune complexes bind to, and are uptaken by, the expanded dendritic cells; and with subsequent processing of the antigen into peptides, the peptide is presented as part of a peptide-MHC complex on the dendritic cell surface. The method of the present invention allows for dendritic cell presentation by MHCI and/or MHCII complexes, depending on the nature of the one or more antigens. Additionally, disclosed is a vaccine which comprises autologous dendritic cells loaded according to the method of the present invention, and autologous, irradiated, DNP-conjugated tumor cells; and a method of vaccinating an individual comprising administering the vaccine to the individual.

This is a nonprovisional application based on earlier co-pending provisional application Serial No. 60/084,041 which is herein incorporated by reference.

### FIELD OF THE INVENTION

The present invention is related to manipulating antigen presenting cells ex vivo. More particularly, the present invention is related to a method for loading antigen presenting cells with antigen for vaccine use or other therapeutic use. Also provided is a vaccine made using the method for loading antigen presenting cells with antigen.

### BACKGROUND OF THE INVENTION

Dendritic cells are specialized antigen presenting cells. Dendritic cells activate resting, naïve T cells by secreting multiple factors that are important for T cell differentiation and growth; capture and process antigen at peripheral sites; and home to areas in lymphoid tissues that are rich in effector T cells. Dendritic cells express high levels of antigen-presenting major histocompatibility complex products (class I and II MHC) as well as accessory molecules that mediate T cell binding. Immature dendritic cells can take up soluble antigens in a fluid phase by macropinocytosis, receptor-mediated endocytosis, or receptor mediated antigen capture. Following uptake, antigens are delivered to endosomal/lysosomal compartments in which antigens are processed by proteases into peptides. The peptides are then loaded onto newly synthesized MHC class II molecules, and the resultant MHC/peptide complexes are transported to the cell surface where they are retained for presentation to T cells.

Because of these features, dendritic cells are currently being evaluated for use in vaccination protocols. More particularly, dendritic cells have been manipulated ex vivo by loading them with antigens. The loaded dendritic cells are then administered in vivo, with the intent that the loaded dendritic cells could then stimulate a powerful cellular immune response against target cells expressing the antigen. Antigen-loaded dendritic cells are being evaluated for clinical immunotherapy. Peptide-pulsed dendritic cells have been shown to be able to induce a cytotoxic lymphocyte (CD8+) antigen-specific protective tumor immunity in against human tumors in an experimental animal model (Celluzi et al., 1996, J. Exp. Med. 183:283-7; Young and Inaba, 1996, J. Exp. Med. 183:7-11); to be successful in vaccinating patients with B cell lymphoma (Hsu et al., 1996, Nature Med. 2:52-8); to be successful in inducing antigen-specific immunity in vaccinated melanoma patients (Nestle et al., 1998, Nature Med. 4:328-332; Hu et al., 1996, Cancer Res. 56:2479-83); to be successful in eliciting a partial beneficial response in prostate cancer patients (Biotechnology Newswatch Apr. 7, 1997); and to vaccinate a multiple myeloma patient resulting in remission (McCann, 1997, J. Natl. Cancer Inst. 89:541-2)

For vaccination purpose, typical populations of dendritic cells utilized include bone marrow-derived dendritic cells, and blood-mobilized dendritic cells. Two methods have been described recently to generate human dendritic cells from hematopoietic precursor cells in peripheral blood. In one method, the relatively rare CD34+ cells in human blood are stimulated with cytokines GM-CSF and TNFα for one to 2 weeks in culture to develop into potent dendritic cells (Caux et al., 1992, Nature 360:258; Caux et al., 1995, J. Immunol. 155:5427; Strunk et al., 1996, Blood 87:1292). Exposure of dendritic cell precursors to GM-CSF is believed to drive maturation of dendritic cells in vitro, and activate a pathway of antigen processing that allows exogenous soluble protein/peptide to be presented by MHC I molecules (Paglia et al., 1996, J. Exp. Med. 183:317-22); whereas TNFα appears to cause a increase in cell surface MHCII and induce preferential formation of MHC/peptide complexes at a high density (Schuler et al., 1997, Int. Arch. Allergy Immunol. 112:317-22).

Another method of mobilizing human dendritic cells from peripheral blood makes use of CD34(−) hematopoietic precursors. Briefly, adherent cells or the light density Percoll fraction from peripheral blood mononuclear cells were depleted of T and B cells, and then cultured in tissue culture medium. These precursors are treated with GM-CSF and IL-4 (Sallusto and Lanzavecchia, 1994, J. Exp. Med. 179:1109-1118). Use of IL-4, as compared to use of TNFα , results in higher stimulatory capacity and the ability to present soluble antigen (Sallusto and Lanzavecchia, 1994, supra). However, this cytokine treatment does not result in stable, mature dendritic cells because removal of these cytokines from the presence of the cells results in loss of the dendritic cell characteristics and morphology (Romani et al., 1996, J. Immunol. Methods 196:137-51). To stabilize maturation of the GM-CSF, IL-4 treated cells, monocyte-conditioned or macrophage-conditioned medium may be added (Romani et al., 1996, supra). A two phase method may be used. A first step involves priming T cell-depleted mononuclear cells in a 6 to 7 day culture in medium supplemented with GM-CSF and IL-4 (IL-13 can be used in place of IL-4). A second step involves inducing differentiation by exposing the culture to macrophage conditioned medium. Such a two step procedure can be used to obtain approximately 1×10⁶ to 3×10⁶ mature dendritic cells from 40 ml of blood (Bender et al., 1996, J. Immunol. Methods 196:121-135). Such cells have been shown to be capable of stimulating CD4+T cell and CD8+ T cell responses. The conditioned medium could not be replaced by adding exogenous cytokines such as IL-1, IL-6, IL-12, IL-15, and TNFα.

Dendritic cells, expanded from peripheral blood, are typically loaded with soluble antigen by incubating (“pulsing”) the antigen and dendritic cells in culture for a sufficient time to allow the dendritic cells to uptake the antigen, and to initiate antigen processing. For example, dendritic cells may be pulsed with antigen for 1 to 2 hours at 37° C. (antigen- 20 ng/ml ovalbumin peptide, Celluzi et al., 1996, supra; antigen- final concentration of 1000 HAU/ml live or inactivated virus, Bender et al., 1996, supra; antigen- 2 μg/10⁶ cells of a melanoma-associated antigen, Hu et al., 1996, Cancer Res. 56:2479-2483). Alternatively, incubation of antigen and dendritic cells may be at room temperature (antigen- 50 μg/ml peptide, Nestle et al., 1998, supra). In an alternate method of pulsing, RNA encoding an antigen may be delivered into dendritic cells, such as by incubating the RNA (25 μg) in the presence of a cationic lipid, and then contacting the RNA-lipid complex with the dendritic cells, e.g., 5×10⁶ cells for 0.5 to 2 hours at 37° C. (Boczkowski et al., 1996, J. Exp. Med. 184: 465-72). In another method of genetically modifying dendritic cells to express and antigen for processing and presentation, retroviral vectors encoding an antigen may be transduced into dendritic cells (Szabolcs et al., 1997, Blood, 90:2160-7; Ashley et al., 1997, J. Exp. Med. 186:1177-82). For example, CD34+ cells were expanded in tissue culture medium supplemented with c-kit-ligand, GM-CSF, and TNFα (with or without flt-3-ligand). For transduction, the expanded cells were co-cultured with a cell line that secretes the retroviral construct. Also, adenoviral vectors have been used to transduce expanded dendritic cells to engineer the transduced dendritic cells to express and present an antigen.

Presentation of soluble antigens by dendritic cells not undergoing cytokine treatment ex vivo can be relatively inefficient in that high concentrations (10⁻⁶M to 10⁻⁷M of antigen are required; whereas cytokine treatment can result in cultured dendritic cells presenting antigen at concentrations of 10⁻¹⁰M (Sallusto and Lanzavecchia, 1994, supra). However, dendritic cells expanded and loaded using current methods in the art have required repeated injections to obtain a anti-tumor effect, indicating that these methods are relatively inefficient (Wan et al., 1997, Human Gene Therapy 8:1355-1367). In that regard, pulsing of dendritic cells by short term exposure to antigen is a relatively inefficient method for loading antigens because the half-life of most MHC-restricted epitopes is only a few to several hours (Wan et al., 1997, supra). While virally transduced dendritic cells may increase the efficiency of antigen presentation, the viruses used present known risks such as an immune response against the virus which could result in the development of neutralizing antibodies, or organ toxicity (e.g., adenoviral vectors can cause liver toxicity) (Wan et al., 1997, supra).

Therefore, there is a need for new methods for expanding dendritic cells, and loading expanded dendritic cells, for use in clinical immunotherapy.

### SUMMARY OF THE INVENTION

The present invention provides methods and compositions for loading dendritic cells ex vivo. Unlike prior methods, the method for loading according to the present invention substantially utilizes immune complex-mediated uptake of an antigen by dendritic cells, with subsequent processing of the antigen into peptides, and presentation of the peptides as part of a peptide-MHC complex on the dendritic cell surface. The antibody having binding specificity for the antigen to be loaded may be human heterologous antibody, or autologous antibody (present in the serum or plasma of the individual to receive the loaded dendritic cells as part of a vaccine protocol). Additionally, the method for loading according to the present invention utilizes target cells, which shed antigen having a target cell-specific epitope specific, for shedding antigen. Thus, unlike prior methods which load peptide only, the method according to the present invention allows for uptake and processing of polypeptides (proteins). Accordingly, the method of the present invention allows for dendritic cell presentation by MHCI and MHCII complexes, depending on the nature of the antigen loaded.

### DETAILED DESCRIPTION OF THE INVENTION

Definitions

The term “dendritic cell” is used herein, for purposes of the specification and claims, to mean an antigen presenting cell which is expanded (proliferated ad differentiated) from a CD34(−) hematopoietic precursor present in peripheral blood mononuclear cell populations in the blood of an individual. Such dendritic cells are specialized in processing antigen, and in immunostimulating naïve, resting T cells in vivo in a process of initiating immune responses of both helper T cells (CD4+) and cytotoxic T cells (CD8+).

The term “antigen” is used herein, for purposes of the specification and claims, to mean a molecule that:

(a) is shed by the target cell (and therefore is soluble);

(b) is also found on the cell surface of the target cell;

(c) is restricted to or uniquely expressed by the target cell (the cell to which the immune response is intended to be directed against), or may express one or more epitopes specific for the target cell; and

(d) is a molecule including, but not limited to, a peptide, polypeptide, protein, glycoprotein, or lipoprotein, wherein following uptake and processing by the dendritic cells, a processed portion (“peptide”) of the antigen is presented by dendritic cells and is capable of inducing an immune response when presented by the dendritic cells. The induced immune response may be cell-mediated and/or humoral.

The term “peptide” is used herein, for purposes of the specification and claims, to mean a series of amino acid residues bonded together (e.g., peptide bonds between the alpha-amino and carbonyl groups of adjacent amino acid residues) which comprise at least one allele-specific motif/epitope such that the peptide will bind an MHC (or MHC-like) allele, and be capable of inducing a T cell response against a desired epitope expressed on the peptide. For example, the peptide is capable of binding to a MHC I type molecule, and being presented to T cells in inducing a cytotoxic T lymphocyte response in vivo against cells which express an antigen which comprises an epitope exhibited by the peptide. “Peptide” is also used herein, for purposes of the specification and claims, to mean a a processed portion of the antigen, following uptake and processing of the antigen by dendritic cells, and as presented by dendritic cells.

The term “target cell” is used herein, for purposes of the specification and claims, to mean a cell expressing a soluble antigen (shed by the target cell, and also found on the cell surface of the target cell) to which the immune response is intended to be directed against. In a preferred embodiment, the target cell is a tumor cell or a virally infected cell. In a more preferred embodiment, the target cells are tumor cells of a tumor type selected from the group consisting of a tumor of ductal epithelial cell origin (e.g., tumors types such as liver, lung, brain, breast, colon, pancreas, stomach, prostate, gastrointestinal tract, or reproductive tract (cervix, ovaries, endometrium etc.)); and a melanoma.

One embodiment of a method of loading dendritic cells ex vivo with one or more soluble antigens produced by target cells according to the present invention comprises:

(a) obtaining peripheral blood mononuclear cells from an individual;

(b) depleting the peripheral blood mononuclear cells of lymphocytes;

(c) contacting and incubating the lymphocyte-depleted peripheral blood mononuclear cells with one or more cytokines to expand the lymphocyte-depleted peripheral blood mononuclear cells to form dendritic cells, and to increase the efficiency of loading of (to maintain or upregulate Fc receptor expression by) the dendritic cells;

(d) incubating the dendritic cells in the presence of target cells and antibody against the one or more antigens of the target cells, and wherein the dendritic cells are cocultured in the presence of, but in the absence of physical contact with, the target cells;

wherein the antibody can combine with the one or more soluble antigens from the target cells in inducing formation of immune complexes, and wherein the dendritic cells load the immune complexes by binding of the immune complexes to Fc receptors on the surface of the dendritic cells, with subsequent processing of the one or more soluble antigens. In an alternative of this embodiment, and as will be more apparent from the description herein, the dendritic cells may be incubated in the presence of medium from the culture of the target cells containing the one or more soluble antigens, and in the presence of antibody.

In another embodiment of a method according to the present invention, already provided is a population of Fc receptor-expressing dendritic cells (as opposed to having to expand peripheral blood mononuclear cells to obtain a population of dendritic cells). A method of loading Fc receptor-expressing dendritic cells ex vivo with one or more soluble antigens produced by target cells comprises: incubating the dendritic cells in the presence of target cells, and antibody against the one or more soluble antigens of the target cells; wherein the dendritic cells are cocultured in the presence of, but in the absence of physical contact with the target cells; wherein the antibody can combine with one or more soluble antigens from the target cells in inducing formation of immune complexes; and wherein the dendritic cells load the immune complexes by binding of the immune complexes to Fc receptors on the surface of the Fc receptor-expressing dendritic cells, with subsequent processing of the one or more soluble antigens. This embodiment of the method may further comprise treating the dendritic cells with one or more cytokines to maintain or upregulate Fc receptor expression by the dendritic cells so as to increase the efficiency of loading. The one or more cytokines may be added to the dendritic cells prior to loading (e.g., before being cocultured with the target cells, and antibody) or during loading (while being co-cultured with the target cells and antibody). In either case, alternatively the dentritic cells may be incubated in the presence of medium from the culture of the target cells containing the one or more soluble antigens, and in the presence of antibody.

Typically, the peripheral blood mononuclear cells (PBMC) are from the individual who is to be receiving the dendritic cells loaded using the method of the present invention; i.e., autologous PBMC. However, the PBMC may be from a biologically compatible source other than autologous PBMC. There are several methods and reagents known to those skilled in the art for isolating PBMC from whole blood. Generally, whole blood (e.g., 50 ml) is drawn from the individual, and collected in a container containing an anticoagulant (e.g., heparin or citrate). The whole blood is then overlayed onto a reagent for gradient separation, and then centrifuged thereby localizing the majority of PBMC in a buffy or interface layer (see, e.g., Freudenthal and Steinman, 1990, Proc. Natl. Acad. Sci. USA 87:7698). The interface layer containing the PBMC (e.g., white blood cells) is harvested. Also, the supernatant plasma is harvested. The PBMC are pelleted by centrifugation (e.g., at 400×g for 25 minutes at room temperature. The plasma (“platelet rich plasma”) is collected in new sterile tubes. To check to see if there was a good cell separation process, the plasma may be sampled and platelets counted. The platelet count can be compared to the PBMC count. Typically, a good separation will result in a ratio of platelets to PBMC of greater than 100:1. The separation procedure may be inadequate if, in the plasma, PBMC are in a concentration of greater than 20 cells/ml; or no platelets are counted. The platelets are then removed from the plasma (e.g., by centrifugation at 2000×g for 20 minutes).

The PBMC pellet is suspended in tissue culture medium (e.g., 10 ml) and kept on ice. There are several methods known in the art to deplete lymphocytes from the PBMC. In one method, PBMC may then be suspended in tissue culture medium, and incubated in tissue culture plates to allow for adherence (e.g., typically, 2 hours at 37° C.). Nonadherent cells, containing primarily lymphocytes and granulocytes, are removed. The adherent cells, containing dendritic cell precursors and other macrophage/monocyte subpopulations, are detached from the plates using methods known to those skilled in the art (e.g., adherent cells may be incubated in phosphate buffered saline, Mg²⁺ and Ca²⁺ free, containing 0.5 mM EDTA at 37° C.). Alternatively, and in a preferred embodiment, PBMC are depleted of CD19+ B cells and CD8+ T cells using an immunomagnetic technique in either a negative selection process or positive selection process of enriching for dendritic cell precursors and other macrophage/monocyte subpopulations. In this preferred embodiment, and as illustrated using a negative selection processs, added to the PBMC suspension are magnetic beads. The magnetic beads are coated with PanB/CD19 antibody and anti-CD8 antibody, and may be washed and suspended in, prior to use, in tissue culture medium (e.g., RPMI containing 1% human serum). Final concentration of the beads to cells may vary, but desirably ranges from 3:1 to 10:1. The beads and PBMC are incubated together with rotation for 30 minutes at 4° C. A magnetic field is then used to remove the magnetic beads (and cells bound thereto) and the lymphocyte-depleted PBMC are harvested. Desirably, the magnetic separation step is repeated to further deplete the PBMC of lymphocytes. The result is a suspension of PBMC depleted of B cells and CD8+ cells. This lymphocyte-depleted PBMC may contain T cells other than CD8+ cells. These remaining T cells may be a source of cytokines for aiding in expanding dendritic cells from the PBMC.

The lymphocyte-depleted PBMC are contacted and incubated with one or more cytokines to expand the dendritic cells. In this step, one or more cytokines which may be used to expand dendritic cells and to maintain or upregulate Fc gamma receptor (e.g., FcγRII) expression by the dendritic cells. The one or more cytokines includes, but is not limited to GM-CSF, IL-4, functional equivalents, and a combination thereof. For example, a functional equivalent of IL-4 for dendritic cell expansion includes IL-13 (Romani et al., 1996, supra). However, it has been reported that TNFαtreatment of dendritic cells downregulates FcγRII expression (Sallusto et al., 1994, J. Exp. Med. 179:1109-1118). Thus, since Fc gamma receptor expression is necessary for the method of loading dendritic cells according to the present invention, treatment of the dendritic cells with a concentration of and incubation time with TNFα, (e.g., 10 ng/ml per 10³ cells for 24 hours) or other cytokine that significantly downregulates FcγRII expression, is not desirable. In one illustration of this embodiment, the lymphocyte-depleted PBMC are adjusted to a concentration of 10⁶ cells/ml in tissue culture medium; and plated at a concentration of about 2.5×10⁶ cells per well in a 6-well tissue culture plate. Added to each well is 0.5 ml of medium which contains a stimulating medium comprising 60% human serum, 6000 U/ml of GM-CSF and 6000 U/ml of human recombinant IL-4 (hrIL-4). Final volume per well is 3 ml, final concentration of serum is 10%, final concentration of GM-CSF is 1000 U/ml, and final concentration of hrIL-4 is 1000 U/ml. The added cytokines are replenished every other day (e.g., days 2, 4 and 6) by removing 0.5 ml of medium from each well, and adding to each well 0.5 ml of fresh stimulating medium. After several days in culture, (e.g. day 8 from initiation of the culture), harvested from the cultures are the non-adherent cells.

At this point, the cultured, expanded lymphocyte-depleted PBMC will have differentiated into distinct cell subpopulations. The adherent cells remaining in culture are primarly macrophages, whereas the nonadherent cells are primarily dendritic cells but also comprise T cells. The nonadherent cells are then pooled, and the volume of the cell suspension is adjusted to 15 ml. T cells in the nonadherent cells are removed from dendritic cells using a method known in the art which may include, but is not limited to, affinity selection, or immunomagnetic methods. For example, T cells in the nonadherent cells are removed from dendritic cells using an immunomagnetic technique in a negative selection process of enriching for dendritic cells. The T cells are removed because in the absence of such cells, MHCI expression increases in the enriched dendritic cells. In this step, added to the nonadherent cells are magnetic beads coated with anti-CD2 antibody. For example, the final concentration of the coated magnetic beads to cells may vary, but desirably ranges from 3:1 to 4:1. The beads and nonadherent cells are incubated together with rotation for 20 to 30 minutes at 4° C. A magnetic field is then used to remove the magnetic beads (and cells bound thereto) and the dendritic cells in the remaining suspension are harvested. Desirably, the magnetic separation step is repeated to further deplete the dendritic cells of T lymphocytes. The dendritic cells are cultured in new wells in the presence of stimulating medium. After a few days in culture (e.g., day 12 from the initial culturing of the lymphocyte-depleted PBMC), 0.5 ml of medium is removed from each well.

In a loading process of the method according to the present invention, the dendritic cells are incubated in the presence of, but in the absence of physical contact with, the target cells; i.e., the dendritic cells are cocultured in physical separation from the target cells. Additionally, added to the coculture is antibody against the antigen(s), thereby inducing the formation of immune complexes with soluble antigen. While there are several ways known to those skilled in the art for achieving such a separation, in one illustration of this embodiment a cell culture system is used in which the dendritic cells and the target cells are each grown in separate chambers. For example, two cell culture chambers are provided, wherein a first chamber fits inside a larger second chamber. The dendritic cells to be loaded may be added into the second chamber, and the target cells may be added to the first chamber, wherein the bottom of the first chamber comprises a microporous membrane, and wherein the target cells grow on the surface of the microporous membrane. The microporous membrane provides pores of sufficient size (e.g., 0.2 to 0.5 micro m) to allow for transport of culture medium containing soluble antigen(s) therethrough, but prevents the passage of cells therethrough. Thus, the membrane allows for physical separation (i.e., in a non-contacting association) between the dendritic cells and the target cells, while allowing for the dendritic cells to be pulsed by soluble antigens shed by the target cells in coculture. In this illustration, cell culture medium in the coculture may be in simultaneous contact with both the dendritic cells and the target cells. Alternatively, where the dendritic cells and the target cells are maintained in separate cell culture chambers, medium from the culture of the target cells containing soluble antigen(s) (“target cell-conditioned medium”) may be controllably circulated into, and may be exchanged for existing medium in, the chamber containing the dendritic cells. The controlled delivery of target cell-conditioned medium pulses the dendritic cells with antigen(s). The medium in the dendritic cell culture may be removed, and exchanged for target cell-conditioned medium, at fixed intervals thereby enhancing the maintenance of the coculture. Commercially available bioreactor systems may be useful in this step of the method of the present invention.

In a specific illustration of the coculturing step and loading process, an insert is provided which (a) contains a chamber for growth of the target cells in physical separation from the dendritic cells; (b) has a membrane to allow for soluble antigens, but not target cells, to pass through the membrane pores; and (c) can be inserted into a well of a 6-well culture plate. In this specific illustration, the target cells are autologous cultured tumor cells. After removing 0.5 ml of media from each well of the dendritic cell culture, inserts are placed into each well. Added to the chamber of each insert is tissue culture medium (e.g., 1 ml of RPMI), the antibody (e.g., heat-inactivated serum from a heterologous or autologous donor; e.g., 20% final concentration of serum), and autologous cultured tumor cells (10⁶ cells). Also added to the culture chamber of the insert may be 0.5 ml of stimulating medium. The cocultures are incubated for a period of time sufficient for the soluble antigens released by the tumor cells to pass through the membrane (e.g., as immune complexes), bind to the dendritic cells via Fc receptor, be uptaken by the dendritic cells, be processed by the dendritic cells, and be presented as part of a peptide-MHC complex. In this illustration, the coculture is maintained for 2 days, the inserts are removed, and the nonadherent, loaded dendritic cells are harvested from the medium contained in each well of the 6-well plate. The yield of loaded dendritic cells should be at least 10⁶ cells per well. The loaded dendritic cells may then be pelleted, washed, and resuspended into a pharmaceutically acceptable carrier (e.g., conventional, nontoxic liquid, solid, or gel carriers) for injection into an individual as part of a vaccine protocol to induce immunity against the target cells in vivo.

It will be appreciated by those skilled in the art that the amount of loaded dendritic cells to be administered (a “therapeutically effective amount”) depends on several factors including, but not limited to, the size and nature (amino acid sequence) of the antigen, the manner of administration, the stage of the disease to be treated, and the immunoresponsiveness of the individual to be treated. For example, a single dose of 10⁸ loaded dendritic cells may be injected intravenously into a patient (see, e.g., Hu et al., 1996, supra). Alternatively, and depending on the disease to be treated, loaded dendritic cells may be administered to the individual via an uninvolved lymph node (see, e.g., Nestle et al., 1998, supra).

### EXAMPLE 1

In this Example, illustrated is a vaccine using the dendritic cells loaded by the method according to the present invention. In this illustration, a vaccine comprised of two components is produced, and the vaccine is a anti-tumor vaccine. A first component comprises a therapeutically effective amount of autologous, living, irradiated melanoma tumor cells which have been conjugated to the hapten dinitrophenol (DNP). DNP-modified tumor vaccines have been shown to improve survival rates over that observed for patients receiving only surgery. However, the a DNP-modified tumor vaccine only induces a limited T cell response, driven by a B cell antigen presentation, consisting of CD4+ cells only. To overcome this limitation, a second component of the vaccine according to the present invention is administered in a therapeutically effective amount. The second component of the vaccine comprises autologous dendritic cells loaded according to the method of the present invention. Such tumor antigen-loaded dendritic cells can increase tumor antigen presentation to the immune system, thus resulting in a substantial anti-tumor response when combined with the DNP-modified tumor component of the vaccine. The resulting combination of such tumor antigen-loaded dendritic cells and DNP-modified tumor cells results in a vaccine that can, uniquely, activate T cells by two pathways. More specifically, the DNP-modified tumor cells, when used to immunize an individual, generate an immune response substantially comprised of CD4+ T cells. The tumor antigen-loaded dendritic cells, produced according to the present invention, can stimulate CD8+ T cells cytotoxic for the tumor (target cells). Thus, the vaccine activates T cells by two pathways: CD4+ cells, and CD8+ cytotoxic cells. Further, the CD4+ cells, generated by administration of a therapeutically effective amount of the DNP-modified tumor cells, can release factors (e.g., IL-2 and other Th1 cytokines) that can augment the anti-tumor effect of the CD8+ cytotoxic cells stimulated by administration of a therapeutically effective amount of the tumor antigen-loaded dendritic cells produced according to the present invention.

In one illustration of the vaccine according to the present invention, tumor cells are obtained from an existing lesion of an individual with metastatic melanoma or recurrent malignant melanoma. For example, a biopsy may be resected from an accessible tumor mass. The tumor tissue is dispersed using mechanical dissociation and enzymatic dissociation (e.g., treatment with collagenase and DNAse), and tumor cells are selected by negative selection in a magnetic immunoaffinity procedure which depletes the tumor tissue of lymphocytes (e.g., using magnetic beads having bound thereto various anti-lymphocyte antibodies). Using such techniques, a desirable number of tumor cells is between about 1×10⁸ cells to about 2×10⁸ cells. A portion of the tumor cell preparation is then irradiated; e.g., by 2500 cGy (centigray). The tumor cells are then washed, suspended in a physiological buffered solution (e.g., saline, or other balanced salt solution), and conjugated with DNP. The conjugation step involves incubating the irradiated tumor cells with DNP for 30 minutes under sterile conditions, followed by washing with a physiological buffered solution. The irradiated, DNP-conjugated tumor cells may be suspended in a physiological solution. For purposes of illustration, a single vaccine dose of this component comprises about 2×10⁷ irradiated, DNP-conjugated tumor cells in about 0.3 ml. This component may further comprise 30 μg/ml of granulocyte-macrophage colony stimulating factor (GM-CSF) (e.g., human recombinant GM-CSF). The GM-CSF, when administered as part of this component of the vaccine, will recruit dendritic cells to the site of the injection; thus, enhancing the presentation of tumor antigen to T cells.

Additionally, blood is taken from the individual, and dendritic cells are expanded therefrom. For example, 50 milliliters of peripheral blood is obtained, and PBMCs are isolated as previously described herein. The PBMCs are then depleted of B cells and CD8+ T cells using an immunomagnetic separation technique. The lymphocyte-depleted PBMCs are then grown ex vivo for 8 days in culture media containing GM-CSF (e.g., 1000 U/ml) and IL-4 (e.g., 1000 U/ml) to expand dendritic cells. On day 8, non-adherent cells are harvested, and transferred to a different culture device (e.g., tissue culture plate) and cultured further for maturation. On day 12, the dendritic cells are pulsed for 48 hours with antigen shed from tumor cells, using the method for loading dendritic cells according to the present invention. Briefly, a portion of the live lymphocyte-depleted tumor cell preparation (e.g., 10⁷ cells), obtained from the individual and prepared as described above, are incubated in a first chamber that is separated by a filter (e.g., 0.2 micrometer pore size) from the dendritic cells (e.g., 10⁶ cells) contained in a second chamber in a total volume of about 5 ml. Generally, the tumor cell to dendritic cell ratio in coculture is about 10:1. Additionally, added to this coculture is serum containing antibody against the tumor (e.g., autologous, heat inactivated serum; in an amount ranging from about 0.5 μg/ml to about 5 μg/ml), and the coculture is incubated for 48 hours. Following the loading process, the dendritic cells may be washed with a physiological buffered solution. The loaded dendritic cells may be suspended in a physiological solution (e.g., in about 0.3 ml of a balanced salt solution). For purposes of illustration, a single vaccine dose of this component comprises about 10⁶ loaded dendritic cells (e.g., about 1×10⁶ to about 8×10⁶ cells). This component may further comprise 30 μg/ml of GM-CSF.

In a method of vaccinating an individual, the two components that comprise the vaccine can be administered to the individual to be vaccinated using a vaccine treatment schedule and route of administration as determined by the skilled medical personnel administering the vaccine. For example, each component (e.g., 0.3 ml) may be divided and administered as three separate injections of 0.1 ml each into the same limb. Desirably, the injections are in contiguous sites on the upper arm or leg, excluding (if possible) limbs ipsilateral to lymph node groups which are presently documented to contain metastatic disease or which have previously undergone lymph node detection. For purposes of illustration, the component comprising about 2×10⁷ irradiated, DNP-conjugated tumor cells in about 0.3 ml and with 30 μg/ml of GM-CSF will be divided into three equal portions with each portion being injected intradermally. These three injections constitute a single treatment with this component. Prior to the first injection (e.g., fourteen days prior), the individual to be vaccinated may be sensitized to DNP by topical application of 1% dinitro-fluorobenzene in acetone-corn oil. After the first treatment (“Day 0”), treatment with additional preparations of this component B may be repeated. An illustrative treatment schedule is shown in Table 1; wherein component A comprises the irradiated, DNP-conjugated tumor cells, and component B comprises dendritic cells loaded according to the present invention. For purposes of illustration, the component comprising 10⁶ dendritic cells loaded according to the present invention in about 0.3 ml and with 30 μg/ml of GM-CSF will be divided into three equal portions with each portion being injected subcutaneously. These three injections constitute a single treatment with this component B. In the treatment schedule illustrated in Table 1, after the first treatment, component A will be given every 28 days; whereas component B will be administered every 14 days; each component being administered for a total of 8 treatments.

TABLE 1
Days

| 0 | 14 | 28 | 42 | 56 | 70 | 84 | 98 | 112 | 140 | 168 | 196 |
|---|---|---|---|---|---|---|---|---|---|---|---|
| A + B | B | A + B | B | A + B | B | A + B | B | A | A | A | A |

While the tumor type illustrated in this example is melanoma, it will be apparent to one skilled in the art from the aforementioned description that a similar vaccine can be made and administered using another tumor type. Such tumor types may include one or more of a tumor type selected from the group consisting of tumor of the liver, the lung, the brain, the breast, the colon, the pancreas, the stomach, the prostate, the gastrointestinal tract, or the reproductive tract.

What is claimed is:

1. A method of loading dendritic cells ex vivo with one or more soluble antigens produced by target cells, wherein the method comprises:
(a) obtaining peripheral blood mononuclear cells from an individual;
(b) depleting the peripheral blood mononuclear cells of lymphocytes;
(c) contacting and incubating the lymphocyte-depleted peripheral blood mononuclear cells with one or more cytokines to expand the lymphocyte-depleted peripheral blood mononuclear cells to form dendritic cells, and to increase the efficiency of loading of the dendritic cells;
(d) incubating the dendritic cells in the presence of target cells, and antibody against the one or more soluble antigens, and wherein the dendritic cells are cocultured in the presence of, but in the absence of physical contact with, the target cells;
wherein the antibody can combine with the one or more soluble antigens in inducing formation of immune complexes, and wherein the dendritic cells load immune complexes by binding of the immune complexes to Fc receptors on the surface of the dendritic cells, and wherein the one or more antigens is processed by the dendritic cells.

2. The method according to claim 1, wherein the one or more cytokines are selected from the group consisting of GM-CSF, IL-4, IL-13, and a combination thereof.

3. The method according to claim 1, wherein the absence of physical contact comprises culturing the dendritic cells in the presence of target-cell conditioned medium comprising medium from a culture of the target cells containing the one or more soluble antigens.

4. The method according to claim 1, wherein the absence of physical contact comprises culturing the dendritic cells in the presence of target cells, wherein a microporous membrane physically separates the dendritic cells from the target cells.

5. The method of claim 1, wherein the antibody comprises serum selected from the group consisting of serum from a autologous donor, and serum from a heterologous donor.

6. The method of claim 1, wherein the target cells are tumor cells of a tumor type selected from the group consisting of a colon tumor, a lung tumor, a brain tumor, a breast tumor, a pancreatic tumor, a stomach tumor, a liver tumor, a prostate tumor, a gastrointestinal tumor, a reproductive tract tumor, and a melanoma.

7. A method of loading Fc receptor-expressing dendritic cells ex vivo with one or more soluble antigens produced by target cells, wherein the method comprises: incubating the dendritic cells in the presence of target cells, and antibody against the one or more soluble antigens; wherein the dendritic cells are cocultured in the presence of, but in the absence of physical contact with, the target cells; wherein the antibody can combine with the one or more soluble antigens in inducing formation of immune complexes; and wherein the dendritic cells load the immune complexes by binding of the immune complexes to Fc receptors on the surface of the Fc receptor-expressing dendritic cells; and wherein the antigen is processed by the dendritic cells.

8. The method according to claim 7, wherein the dendritic cells are incubated with one or more cytokines selected from the group consisting of GM-CSF, IL-4, IL-13, and a combination thereof.

9. The method according to claim 7, wherein the absence of physical contact comprises culturing the dendritic cells in the presence of target-cell conditioned medium comprising medium from a culture of the target cells containing the one or more soluble antigens.

10. The method according to claim 7, wherein the absence of physical contact comprises culturing the dendritic cells in the presence of target cells, wherein a microporous membrane physically separates the dendritic cells from the target cells.

11. The method of claim 7, wherein the antibody comprises serum selected from the group consisting of serum from a autologous donor, and serum from a heterologous donor.

12. The method of claim 7, wherein the target cells are tumor cells of a tumor type selected from the group consisting of a colon tumor, a lung tumor, a brain tumor, a breast tumor, a pancreatic tumor, a stomach tumor, a liver tumor, a prostate tumor, a gastrointestinal tumor, a reproductive tract tumor, and a melanoma.

13. Dendritic cells loaded according to the method of claim 1.

14. Dendritic cells loaded according to the method of claim 6.

15. Dendritic cells loaded according to the method of claim 7.

16. Dendritic cells loaded according to the method of claim 12.

17. A vaccine comprised of two components, wherein a first component comprises a therapeutically effective amount of autologous, living, irradiated target cells which have been conjugated to dinitrophenol; and a second component which comprises a therapeutically effective amount of the dendritic cells according to claim 13 which are autologous.

18. A vaccine comprised of two components, wherein a first component comprises a therapeutically effective amount of autologous, living, irradiated target cells which have been conjugated to dinitrophenol; and a second component which comprises a therapeutically effective amount of the dendritic cells according to claim 14 which are autologous.

19. A vaccine comprised of two components, wherein a first component comprises a therapeutically effective amount of autologous, living, irradiated target cells which have been conjugated to dinitrophenol; and a second component which comprises a therapeutically effective amount of the dendritic cells according to claim 15 which are autologous.

20. A vaccine comprised of two components, wherein a first component comprises a therapeutically effective amount of autologous, living, irradiated target cells which have been conjugated to dinitrophenol; and a second component which comprises a therapeutically effective amount of the dendritic cells according to claim 16 which are autologous.

21. A method of vaccinating an individual comprising administering to the individual the vaccine according to claim 17.

22. A method of vaccinating an individual comprising administering to the individual the vaccine according to claim 18.

23. A method of vaccinating an individual comprising administering to the individual the vaccine according to claim 19.

24. A method of vaccinating an individual comprising administering to the individual the vaccine according to claim 20.
